(19) United States
(12) Patent Application Publication
(10) Pub. No.: US 2026/0088166 A1
(43) Pub. Date: Mar. 26, 2026

(54) CLIENT-ADVISOR PORTAL SYSTEMS AND METHODS

(71) Applicant: Vell LLC, Jupiter, FL (US)

(72) Inventors: Michael Gioan, Huntington Beach, CA (US); Ken Grey, Jupiter, FL (US)

(73) Assignee: Vell LLC, Jupiter, FL (US)

(21) Appl. No.: 19/336,157

(22) Filed: Sep. 22, 2025

Related U.S. Application Data
(60) Provisional application No. 63/698,690, filed on Sep. 25, 2024.

(51) Int. Cl. G16H 40/67; G16H 50/20; G16H 80/00

(52) U.S. Cl. CPC G16H 40/67; G16H 50/20; G16H 80/00

(57) ABSTRACT

Systems and methods for active monitoring of clients by advisors are described. Clients may include patients, and advisors may include medical practitioners. Active monitoring of clients may include reading sensor data from devices associated with a client and then sending notifications to the client. The notifications may be based on a set of alert rules based on a health management profile of the patient. The alert rules may be modified by the advisor. Embodiments may include systems and methods to analyze the effectiveness of active monitoring on clients. Systems and methods may involve recommendations of whether active monitoring can benefit a client.

This application claims the benefit of priority of U.S. Provisional Patent Application No. 63/698,690, entitled “CLIENT-ADVISOR PORTAL SYSTEMS AND METHODS,” filed Sep. 25, 2024, the entire contents of which are incorporated herein by reference for all purposes.

### BACKGROUND

Applications that monitor health, finances, and educational progress have increased in popularity. As an example, health monitoring applications have become increasingly popular as tools for individuals to track various aspects of their health and well-being. These applications typically collect data from wearable devices, such as fitness trackers and smartwatches, as well as from user inputs regarding diet, exercise, sleep, and other lifestyle factors. By providing users with real-time feedback and trends over time, these applications aim to empower individuals to make informed decisions about their health and to adopt healthier habits. The widespread adoption of such applications highlights their potential to play a significant role in preventive health care, offering personalized insights that can contribute to better health outcomes.

One significant limitation of many health monitoring applications is the lack of integration with healthcare providers, particularly the limited ability for physicians to directly control or influence the data collected and the recommendations provided by these apps. Most health monitoring applications are designed for direct consumer use, with minimal input from medical professionals. As a result, the recommendations generated by the app may not be tailored to an individual's specific medical needs. This disconnect can lead to inconsistencies in care, where the guidance offered by the application may conflict with a physician's advice, potentially undermining the effectiveness of both. Furthermore, without physician oversight, critical health data may not be adequately monitored, and early warning signs of serious conditions could be missed. Integrating physician control into health monitoring applications could enhance their reliability, ensure better alignment with personalized treatment plans, and ultimately lead to more effective health management.

Additionally, there remains a significant question regarding their actual effectiveness in improving health outcomes. While these applications generate a wealth of data and can provide users with actionable insights, it is not always clear whether users consistently follow the recommendations provided or whether these recommendations lead to measurable improvements in health. Additionally, there is a need to understand the long-term impact of these applications on user behavior and health metrics. Determining the effectiveness of health monitoring applications is crucial for validating their utility as tools in preventive medicine and for guiding future development to enhance their impact.

Applications are not limited to the healthcare concepts. Systems and methods described herein address the integration of the monitoring applications, the determination of the effectiveness of active monitoring apps, and other improvements.

### BRIEF SUMMARY

Embodiments of the present invention include systems and methods for active monitoring of clients by advisors. Clients may include patients, and advisors may include medical practitioners. Active monitoring of clients may include reading sensor data from devices associated with a client and then sending notifications to the client. The notifications may be based on a set of alert rules based on a health management profile of the patient. The alert rules may be modified by the advisor. Embodiments may include systems and methods to analyze the effectiveness of active monitoring on clients. Systems and methods may involve recommendations of whether active monitoring can benefit a client.

A computer-implemented method may include receiving, into a model, values of characteristics associated with a subject. The model may be configured to process data for a plurality of clients and to evaluate an effectiveness of a monitoring program. The plurality of clients may include a monitored subset of clients who have been subject to the monitoring program. The monitoring program may be configured to send notifications to one or more advisors when sensor data satisfies an alert rule for a client. The alert rule for the client may be customizable by the one or more advisors. The method may include generating, using the model, a classification of the effectiveness of the monitoring program for the subject. The method may include inputting a status of the subject into the monitoring program using the classification. The status may indicate whether to implement the monitoring program for the subject.

Systems and computer-readable medium related to executing the method are also described.

### TERMS

The term “classification” as used herein refers to any number(s) or other characters(s) that are associated with the effectiveness of the monitoring program. For example, a “+” symbol (or the word “positive”) could signify that the monitoring program is effective. The classification can be binary (e.g., positive or negative) or have more levels of classification (e.g., a scale from 1 to 10 or 0 to 1), including probabilities.

The terms “cutoff” and “threshold” refer to predetermined numbers used in an operation. For example, a threshold value may be a value above or below which a particular classification applies. Either of these terms can be used in either of these contexts. A cutoff or threshold may be “a reference value” or derived from a reference value that is representative of a particular classification or discriminates between two or more classifications. A cutoff may be predetermined with or without reference to the subject. For example, cutoffs may be chosen based on the age or sex of the tested subject. A cutoff may be chosen after and based on output of the test data. A reference value can be selected as representative of one classification (e.g., a mean) or a value that is between two clusters of the metrics (e.g., chosen to obtain a desired outcome, sensitivity, and/or specificity). Any of these terms can be used in any of these contexts.

A “machine learning model” (ML model) can refer to a software module configured to be run on one or more processors to provide a classification. An ML model can be generated using sample data (e.g., training data) to make predictions on test data. One example is an unsupervised learning model. Another example type of model is supervised learning that can be used with embodiments of the present disclosure. Example supervised learning models may include different approaches and algorithms including analytical learning, statistical models, artificial neural network, backpropagation, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naïve Bayes classifier, maximum entropy classifier, conditional random field, nearest neighbor algorithm, probably approximately correct learning (PAC) learning, ripple down rules, a knowledge acquisition methodology, symbolic machine learning algorithms, subsymbolic machine learning algorithms, minimum complexity machines (MCM), random forests, ensembles of classifiers, ordinal classification, data pre-processing, handling imbalanced datasets, statistical relational learning, or Proaftn, a multicriteria classification algorithm. The model may include linear regression, logistic regression, deep recurrent neural network (e.g., long short term memory, LSTM), hidden Markov model (HMM), linear discriminant analysis (LDA), k-means clustering, density-based spatial clustering of applications with noise (DBSCAN), random forest algorithm, support vector machine (SVM), or any model described herein. Supervised learning models can be trained in various ways using various cost/loss functions that define the error from the known label (e.g., least squares and absolute difference from known classification) and various optimization techniques, e.g., using backpropagation, steepest descent, conjugate gradient, and Newton and quasi-Newton techniques.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” or “approximately” can mean within an order of magnitude, within 5-fold, and more preferably within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the embodiments of the present disclosure, some potential and exemplary methods and materials may now be described.

### BRIEF DESCRIPTION OF THE DRAWINGS

To easily identify the discussion of any particular element or act, the most significant digit or digits in a reference number refer to the figure number in which that element is first introduced.

FIG. 1 illustrates a graphical user interface according to embodiments of the present invention.

FIG. 2 illustrates a graphical user interface according to embodiments of the present invention.

FIG. 3 illustrates a graphical user interface according to embodiments of the present invention.

FIG. 4 illustrates a graphical user interface according to embodiments of the present invention.

FIG. 5 illustrates a graphical user interface according to embodiments of the present invention.

FIG. 6 illustrates a system according to embodiments of the present invention.

FIG. 7 illustrates an example of interactions using a health monitoring application according to embodiments of the present invention.

FIG. 8 illustrates integration of a health monitoring application according to embodiments of the present invention.

FIG. 9 illustrates an aspect of the subject matter in accordance with one embodiment.

FIG. 10 illustrates a method 1000 in accordance with one embodiment.

FIG. 11 illustrates a block diagram illustrating using one or more machine learning models according to embodiments of the present invention.

FIG. 12 illustrates a computing device according to embodiments of the present invention.

### DETAILED DESCRIPTION

In a client-advisor relationship, both parties seek for improved outcomes for the client. This is true for many different types of advisors, including healthcare advisors, business advisors, educational advisors, financial advisors, and legal advisors. Clients and advisors may have regular meetings to check on progress. However, these meetings may not be at a frequency to monitor progress effectively. Monitoring applications, which may be on a mobile phone, may be one way to achieve improved outcomes. As an example, consider the physician-patient client-advisor relationship.

Typical health monitoring applications are limited by little or no coordination with care providers and little or no understanding regarding the effectiveness of such programs. Many users of such applications may have high initial interest in using the application. However, after time, interest wanes and users of applications may not use the application and/or may ignore notifications from the application. Such health monitoring applications may be generalized for the user with little or no customization of the particular user's health profile.

Embodiments of the present invention bring the care provider into a health care application. The care provider may be able to manage alert rules for a patient or a plurality of patients. The alert rules may be determined for specific health management programs (e.g., blood sugar reduction, diabetes management, cardiovascular health). Alerts may notify the patient when condition(s) are satisfied. Alerts may be configured to correct detrimental or non-beneficial behavior. Alerts may also be configured to encourage beneficial behavior or reward specific health outcomes. The particular alert rules may be determined by the care provider with or without assistance of a machine learning model.

Embodiments may also include determining whether a particular patient is likely to have improved health outcomes with an active monitoring program. An active monitoring program may refer to a program where the care provider can set or customize alerts for a patient. For example, data regarding the particular patient may be input into a model. The data may include demographic data, health profile data (e.g., provided by user and/or care provider), and/or health application data. The model may be defined based on similar data from a plurality of patients. The model may output whether active monitoring of the particular patient is likely to be effective. An effective active monitoring program may be one in which patient health outcomes improve. An effective program may result in the patient reaching certain goals, which may be quantitative (e.g., target A1C level, target blood pressure, target resting heart rate, target bone density) or qualitative (e.g., patient's self-evaluation of physical or mental health, care provider's assessment of patient health). In some embodiments, the goal may be maintenance of a certain level rather than an improvement to reach the target.

The care provider and/or the patient may decide to implement the active monitoring program. In embodiments, the active monitoring program may be considered a treatment for the patient. For example, the active monitoring program may prescribe certain behaviors, use of certain medications, and/or care routines.

The described embodiments may provide a specific improvement to the functioning of computer systems and graphical user interfaces (GUIs) by dynamically rearranging health-related monitoring elements (e.g., alert rules, notifications, commendations, warnings, and appointment requests) based on determined usage and effectiveness criteria. Unlike generic data presentation, the claimed methods integrate clinical relevance and adaptive user interaction into the arrangement of interface elements, thereby reducing the time, cognitive load, and resource usage required for patients and advisors to identify and act on important information.

The determination of usage may involve tracking sensor data, monitoring advisor and patient interactions across devices, and updating interface arrangements in real time based on outcomes and machine learning classifications. These operations may involve processing of electronic health data and sensor-derived metrics that are not practically performed in the human mind. The improvements may arise from the use of computer technology to automatically integrate multi-factor health data with advisor-driven priorities.

The rearrangement of interface elements may not be a stand-alone concept of organizing information but may be integrated into the broader patient monitoring and advisor portal system. The improved GUI may ensure that critical alerts and commendations are surfaced in a context-dependent manner, improving compliance and enabling timely clinical interventions. By automatically elevating clinically significant alerts while deprioritizing less relevant items, the system may be a solution to a technological problem in health monitoring.

Conventional monitoring applications display alerts and notifications in static or generic orderings, such as chronological lists, without regard to clinical relevance or prior patient response. In contrast, the claimed system may involve adaptive rearrangement logic driven by both sensor data and advisor input, thereby yielding a tangible improvement in health outcomes and usability. This specific arrangement may be a technical solution to a technical problem.

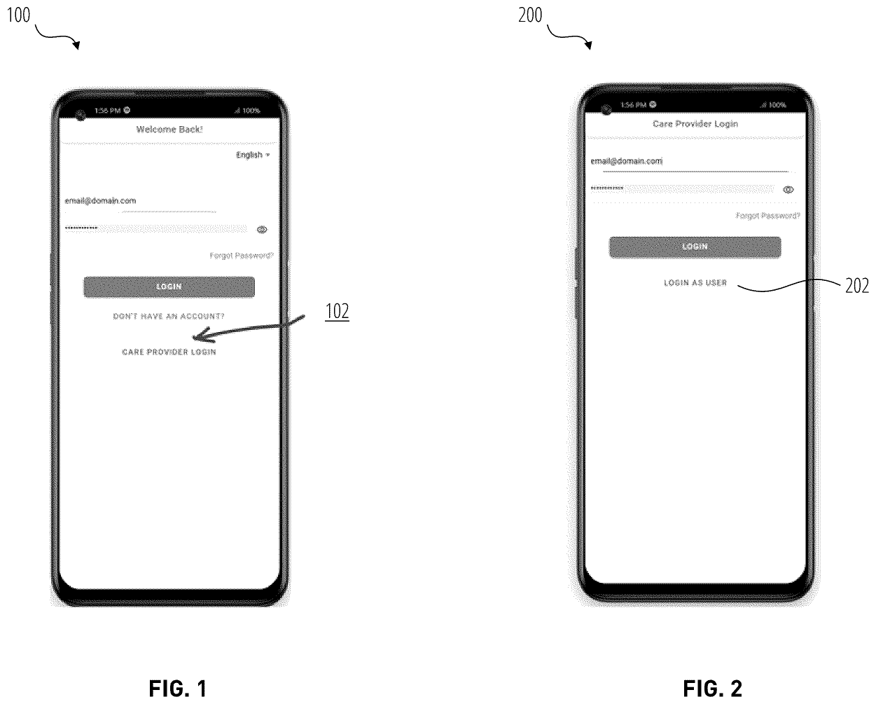

FIG. 1 shows a user login screen 100 for graphical user interface (GUI) for a health monitoring application with the option for active monitoring. A user may include a patient of a medical practitioner. The user may be interested in improving their health outcomes. The user may have a disorder or disease that requires management or care. User login screen 100 may include a care provider login option 102.

FIG. 2 shows a provider login screen 200 for a GUI for the health monitoring application. The provider login screen 200 may be displayed after care provider login option 102 is selected. Provider login screen 200 includes user login option 202, which when selected, may bring up user login screen 100. Provider login screen 200 may be for medical practitioners (e.g., physicians, physician assistants, nurses, holistic medicine practitioners) or designees of medical practitioners (e.g., administrators, health insurance agents, other medical staff). Provider access may be limited to organizations or personnel verified to be a medical provider.

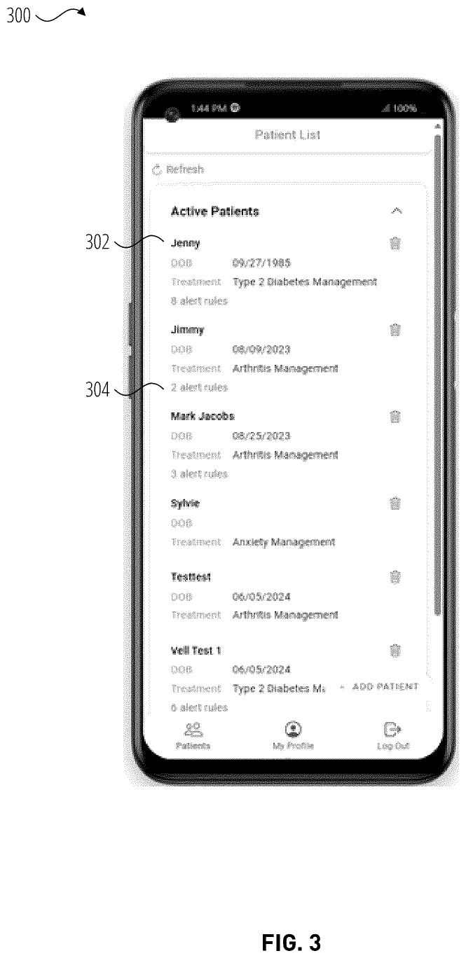

FIG. 3 shows patient list screen 300, which may be viewable by a care provider. Patient list screen 300 may include patient information 302. Examples of patient information 302 include name, date of birth, age, gender, patient treatment plan, or patient disorder. Patient list screen 300 may also include an option to select alert rules 304.

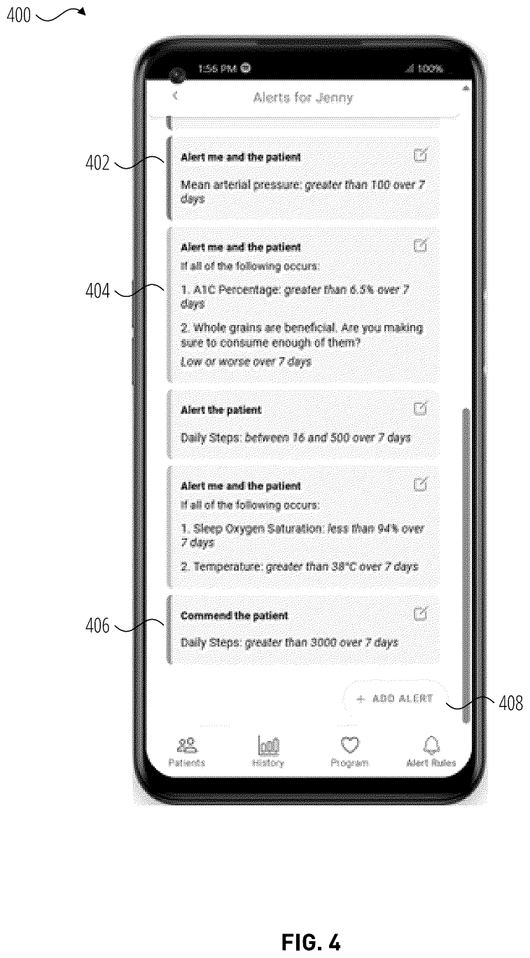

FIG. 4 shows alert screen 400, which may be displayed after alert rules 304 is selected. Alert screen 400 may list different alert rules. Alert screen 400 shows prioritized alert 402. Prioritized alert 402 indicates that the patient and the care provider will be alerted when the mean arterial pressure is greater than 100 for over 7 days. Prioritized alert 402 may be denoted with a specific color (e.g., red) and/or may appear at the top of alert screen 400. Alert screen 400 may also include alerts that may not be prioritized.

An alert may include a multi-condition alert 404. A multi-condition alert requires multiple conditions to be satisfied before a notification is sent. For example, multi-condition alert 404 will alert the care provider and the patient when both the A1C percentage is greater than a threshold for a certain duration and if the user is not indicating enough whole grain intake.

Alert screen 400 may also include a commendation 406, which generates a notification praising the patient. As an example, if daily steps are greater than a threshold for a certain duration, then the application may send a notification stating the achievement. Other commendations may be possible including digital badges or financial incentives.

Alert screen 400 may include add alert option 408. Add alert option 408 may allow for the care provider to set conditions and actions resulting when the conditions are fulfilled.

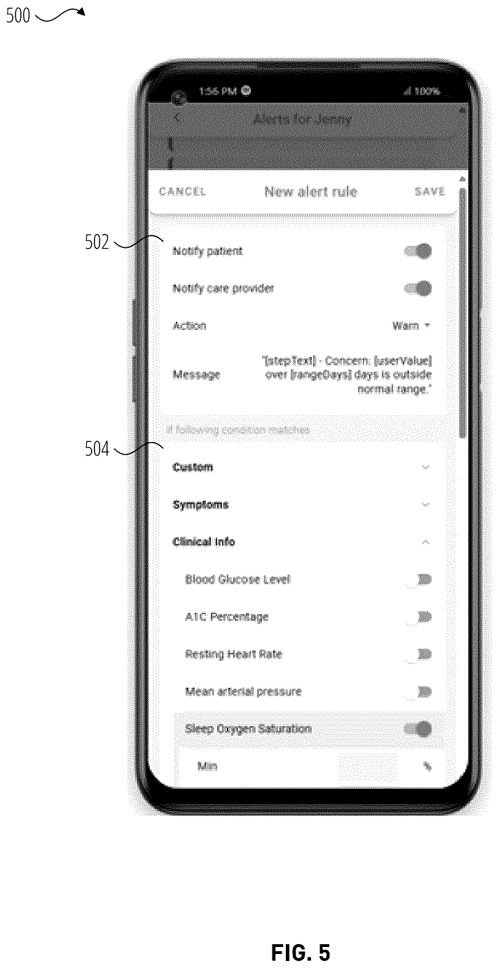

FIG. 5 shows rule creation screen 500, which may be invoked after add alert option 408 is selected. Rule creation screen 500 may be an interface for a care provider to set up alert rules. Rule creation screen 500 may include notification options 502. Notification options 502 may allow for notifications to the patient and/or care provider. Notification options 502 may also include the type of action for the notification (e.g., warn or commend). Notification options 502 may include the message to be communicated in the notification. The message may include one or more variables related to the type of alert. For example, the variables may indicate the condition or conditions that were satisfied.

Rule creation screen 500 may include conditions configuration 504. Conditions configuration 504 may list different symptoms or clinical information to be satisfied for an alert.

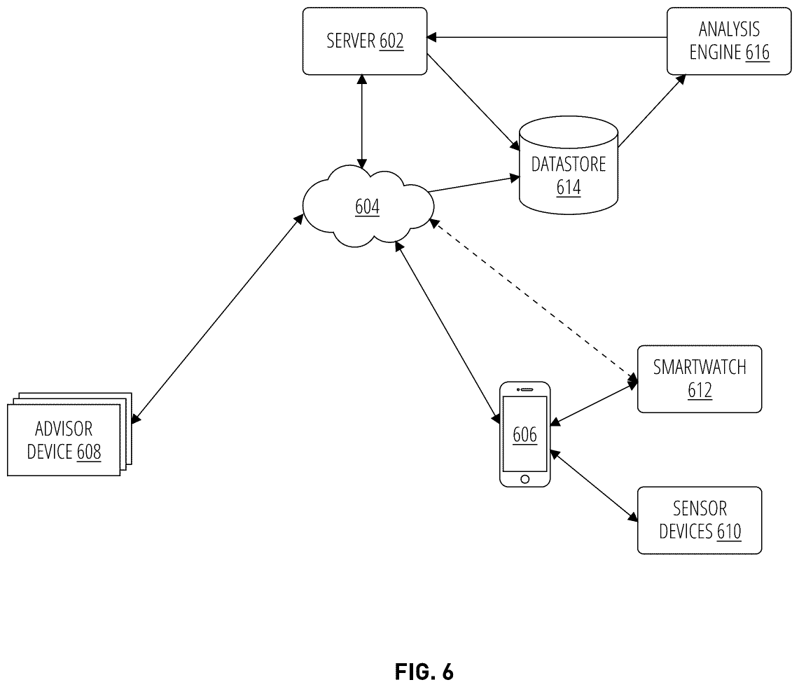

As shown in FIG. 6, user device 606 may be connected to smartwatch 612 and/or sensor devices 610. User device 606 may be a mobile phone, tablet, computer, smartwatch, or other suitable device. User device 606 may include an active monitoring application. Smartwatch 612 and/or sensor devices 610 can include one or more sensors. Sensors may provide data on heart rate, blood pressure, blood oxygen, blood sugar (e.g., A1C, glucose), activity (e.g., steps), body temperature, or other suitable health-related metrics. Smartwatch 612 and/or sensor devices 610 may transmit sensor data associated with the user to user device 606. In some embodiments, smartwatch 612 and/or sensor devices 610 may transmit sensor data to server 602 via network 604. User device 606 may store the sensor data and/or transmit the sensor data to server 602 and/or server 602 through network 604.

Advisor device 608 may be a mobile phone, tablet, computer, or other suitable device. Advisor device 608 may transmit information to server 602 through network 604. The information transmitted to server 602 may result in server 602 updating an application on user device 606.

Server 602 may be server 706 or any server described herein. User device 606 may be operated by client 704. User device 606 may include an application that sends notifications to the user. advisor device 608 may be operated by advisor 702. Advisor device 608 may include an application that sends notifications to the medical practitioner. The notifications sent to the user and/or advisor may be determined by rules, which may be marked for selection on server 602.

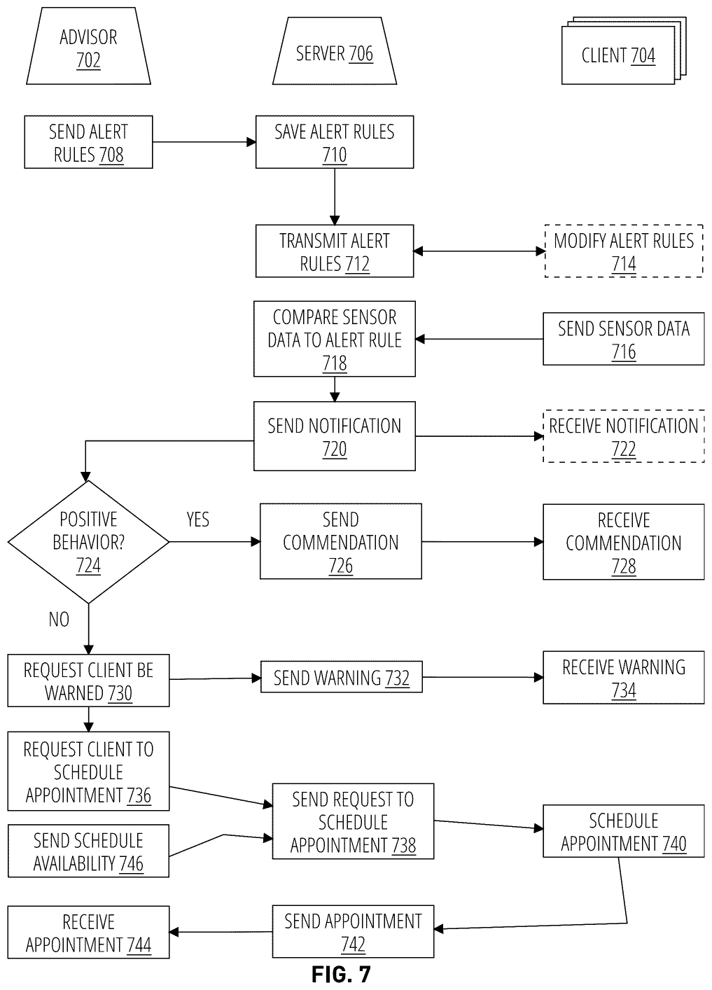

Data may be stored in datastore 614, which may be a data base, data lake, data mart, or any suitable datastore. Data may be from server 602 and/or network 604. Data may include any sensor data from user device 606, smartwatch 612, or sensor devices 610. Additionally, data in datastore 614 may include communications between advisor device 608 and user device 606. Notifications on user device 606 and dispositions (e.g., acknowledged, ignored, dismissed) of those notifications may be stored in datastore 614. Actions described with FIG. 7 may also be stored in datastore 614. Datastore 614 may store data for not just a single user device 606 and a single advisor device 608 but may store data for a plurality of user devices and/or a plurality of medical practitioner devices. For example, the plurality may include 100 to 1,000, 1,000 to 10,000, 10,000 to 100,000, 100,000 to 1 million, or over 1 million devices.

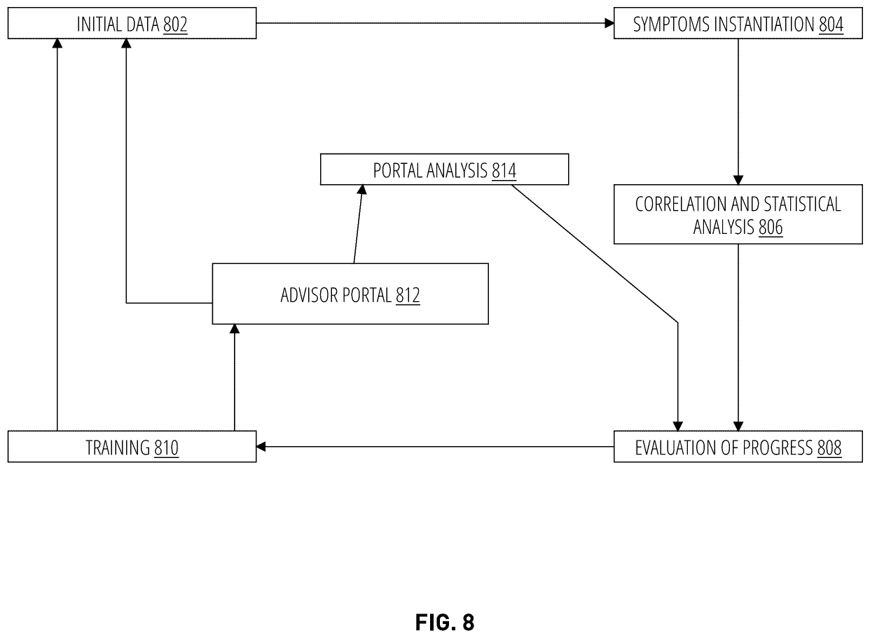

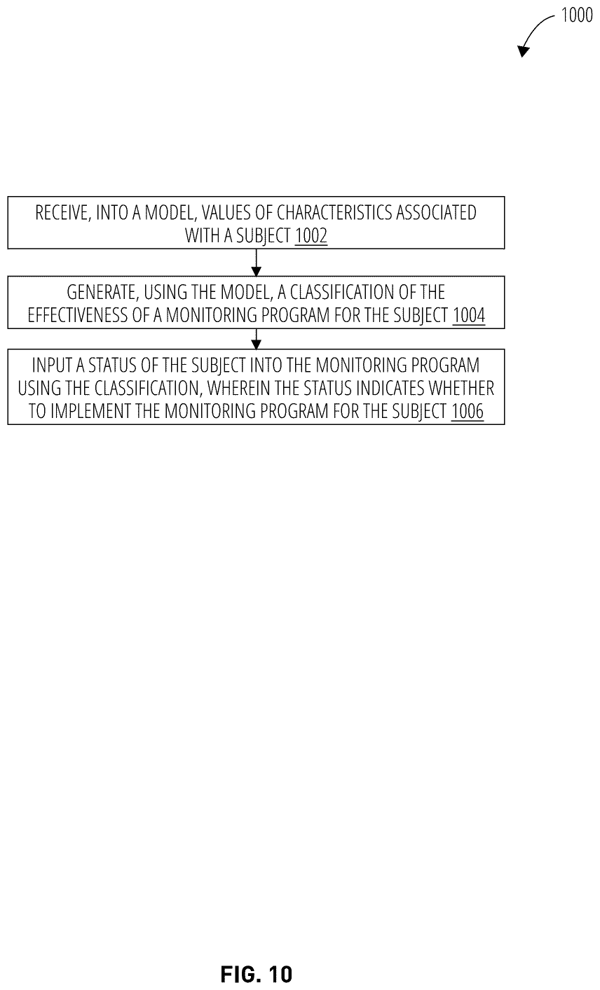

Analysis engine 616 may analyze data stored in datastore 614. Analysis engine 616 may be a computing device 1200. Analysis engine 616 may use statistical analysis or machine learning techniques. Analysis engine 616 may be used in portal analysis 814 of FIG. 8. Analysis engine 616 may determine whether the application on the user device 606, the application on the advisor device 608, and/or the communications from server 602 are effective in improving outcomes for patients. The analysis is described in more detail with FIG. 8 and FIG. 10. Based on the analysis, analysis engine 616 can update server 602 with information on the effectiveness of health applications on devices. With the information, server 602 may instruct advisor device 608 and/or user device 606 to prompt the medical practitioner or user, respectively, to implement or not implement active monitoring. In some embodiments, server 602 may suggest certain types or frequencies of notifications.

The machine-learning models may be trained using training data received or derived from data from datastore 614. In some instances, a processor (e.g., processor 1224) may define training thresholds based on the particular machine-learning model being trained. The training thresholds may correspond to a quantity of training data, a type of training data, and/or the like. For example, if the quantity of training data is less than a threshold quantity of training data or does not correspond to a threshold training data type, additional data may be generated and/or identified that can be used to augment the training data. Processor 1224 may generate additional data procedurally (e.g., using semi-automated or automated software processes, etc.), manually, a combination thereof, or the like.

Processor 1224 may determined a set of feature vectors from the training data. The set of feature vectors may be used to train the particular machine-learning model. Machine-learning models may be trained using supervised training, supervised training, semi-supervised training, reinforcement training, combinations thereof, or the like. The training phase for a particular machine-learning model may be based on a target accuracy of the machine-learning model. For example, a machine-learning model may be trained until the target accuracy is reached. In some instances, the machine-learning model may be trained until the target accuracy is reached or one or more other criteria is met (e.g., such as time, efficiency, and/or the like). For example, if a threshold time interval expires before the machine-learning model reaches the target accuracy, then the training phase may be restarted (e.g., with a new machine-learning model) or the training data may be analyzed to determine if the training data is sufficient in quantity and/or type to train the machine-learning model.

Once trained, the machine-learning models may be executed (e.g., by processor 1224, computing device 1200, etc.) to generate predictions for a user and/or user device. For example, user device 606 may execute a health monitoring application. User device 606 may transmit a request for health monitoring to server 602. The request may include a user identifier of user device 606, an identification of one or more symptoms, and an indication of a management program type. Server 602 may receive the request and identify data associated with the user of user device 606 in datastore 614. Alternatively, or additionally, the data associated with the user of user device 606 may be transmitted by user device 606 and/or one or more other devices with the request. Analysis engine 616 may identify one or more machine-learning models and/or an ensemble model of machine-learning models based on the one or more symptoms, the management program type, and/or the data associated with the user. Features may be extracted from the data associated with the user and define a feature vector based on the identified one or more machine-learning models and/or ensemble model, the one or more symptoms, the management program type, and/or the like. Analysis engine 616 may execute the identified one or more machine-learning models and/or ensemble model using the feature vector as input.

The machine-learning models and/or ensemble model may generate a classification of the effectiveness of an active monitoring program for the user. The classification may be whether an active monitoring program is effective. The classification may include characteristics of an active monitoring program, which may include the frequency of notifications, the type of monitoring, the type of sensors, the conditions for an alert, or the management program type.

The classification may be communicated to a medical practitioner of advisor device 608 and/or the user of user device 606 through the respective devices. The medical practitioner and/or the user may determine whether to enable the active monitoring program for the user. The instruction to enable the active monitoring program may be sent by advisor device 608 and/or user device 606 through network 604 to server 602. advisor device 608 and/or user device 606 may offer the medical practitioner and/or user to accept the recommended active monitoring program as provided by analysis engine 616 or to customize characteristics of the active monitoring program. Datastore 614 may be updated to include the status of the active monitoring program for the specific user.

The use of the active monitoring program on user device 606 may provide additional feedback data. In some instances, the feedback data may be passed to the one or more machine-learning models and/or ensemble model that generated the classification of the effectiveness for reinforcement learning. In those instances, analysis engine 616 may analyze the feedback to determine the suitability of the feedback for reinforcement learning (e.g., based on content, format, a current accuracy metric of the one or more machine-learning models and/or ensemble model, etc.). If the feedback is determined to be suitable, then features may be extracted from the feedback that can be passed to the one or more machine-learning models and/or ensemble model for the reinforcement learning.

In some instances, analysis engine 616 may re-analyze data in datastore 614, which may or may not include data of the specific user. Analysis engine 616 may generate a second classification of the effectiveness of an active monitoring program for the specific user. If the second classification differs from the first classification, server 602 may communicate the change to the medical practitioner and/or user. The medical practitioner and/or user can enable or disable the active monitoring program based on the second classification.

FIG. 7 shows an example of the interaction between advisor 702, server 706, and client 704. Advisor 702 may be a medical practitioner (e.g., physician, physician's assistant, nurse, physical therapist, or clinic/hospital staff), a fitness coach, a career coach, or a life coach. The steps illustrated for advisor 702 are performed using a device, including advisor device 608. Server 706 may be server 602 or a computing device 1200. Server 706 may be an on-site or cloud server. client 704 may be an individual receiving care from advisor 702. The steps illustrated for client 704 are performed using a device, including user device 606. FIG. 7 shows a single patient for simplicity. However, the interaction may involve a plurality of patients, including from 10 to 50, 50 to 100, 100 to 200, 200 to 500, 500 to 1,000, or over 1,000 patients.

At block 708, advisor 702 sends alert rules to server 706. As explained with FIG. 6, advisor 702 may determine to send alert rules after receiving a classification of the effectiveness of an active monitoring program, where the classification is generated by analysis engine 616. The alert rules may be customized for client 704. Alert rules may include conditions for an alert and recipients for the alert. advisor 702 may configure the alert rules before sending. As described herein, advisor 702 may select profiles and/or alter conditions for alert rules before sending. Altering the conditions may include modifying limits in the conditions for alert rules. Altering the conditions may include adding conditions, removing conditions, or changing an alert to require a subset of conditions instead of all conditions (or vice versa). advisor 702 may also grant or deny permissions for client 704 to modify alert rules.

At block 710, server 706 may save the alert rules. The alert rule may be saved to a computer-readable storage medium (e.g., datastore 614).

At block 712, server 706 may transmit the alert rules to client 704. The alert rules may be transmitted over a network by any suitable communication means. The transmitted alert rules may result in an application on a user device 606 of client 704 presenting the alert rules.

At block 714, client 704 may optionally modify the transmitted alert rules. Client 704 may modify alert rule notifications. For example, client 704 may disable notifications for certain alert rules. The notifications may be disabled for advisor 702 and/or client 704. In some embodiments, client 704 may modify the conditions for the alerts, as described for advisor 702 in block 708. client 704 may modify the alert rules provided that advisor 702 has granted permission to client 704 to modify the alert rules.

In some embodiments, advisor 702 may modify alert rule notifications using information from their specific relationship with client 704. In a medical context, a medical practitioner may know that the patient has a specific physiology or pathology and modify the alert rule notification accordingly. As an example, a patient may have tolerance to a glucose level or high blood pressure, and while these levels may be out-of-spec for a typical patient, they may not be an issue with this particular patient. Hence, the medical practitioner may turn off an alert rule notification or change the range based on a patient's specific medical history and/or etiology of condition, including hereditary, cultural or genetic variations.

In a financial context, a financial advisor may be aware of certain life status events that would affect spending habits and trends. For example, a client may be undergoing medical treatment, which would increase spending beyond historical or typical levels. Rather than add to the client's stress with the notifications about spending, the financial advisor may alter or turn off alert rule notifications. Similarly, as a client enters retirement, spending may increase to surpass income, which may not be as much of a cause for concern compared to the client's prime working years.

At block 716, client 704 may send sensor data to server 706. Sensor data may be collected by sensor devices 610, smartwatch 612, or any device described herein. Sensor data may be any type of data described herein.

At block 718, server 706 may compare sensor data to the alert rules. The comparison may involve the sensor data being compared to one or more thresholds in condition(s) of the alert rule to see if the condition(s) are satisfied. For example, the sensor data may be compared to a threshold to determine whether the sensor data is below or above the threshold. Either below or above the threshold may be considered to satisfy or partly satisfy the condition. In some embodiments, comparing the sensor data to the alert rule may involve determining whether sensor data is statistically the same or different as reference data, which may be past data from client 704 or data from control subjects. The comparison may be a statistical test, including Two-Sample T-Test, Paired T-Test, Z-Test, Mann-Whitney U Test, Wilcoxon Signed-Rank Test, Kolmogorov-Smirnov Test, Levene's Test, Bartlett's Test, Cumulative Sum Control Chart (CUSUM), Exponentially Weighted Moving Average (EWMA), or Hotelling's T-Squared Test. Although FIG. 7 shows block 718 as being performed by server 706, block 718 may be performed by client 704 using user device 606.

At block 720, server 706 sends out a notification provided that the comparison in block 718 shows that the conditions in the alert rule are satisfied. The notifications may be sent as an email, text message, and/or push notification. In some embodiments, the notification may be sent to another device, which may provide a signal. For example, the notification may be sent to smartwatch 612 or any device, which may provide haptic feedback or a visual or audio signal.

At block 722, client 704 may optionally receive the notification. In some embodiments, the notification may not be sent to client 704. The notification may provide client 704 with an option to confirm receipt of the notification.

At decision block 724, different paths are performed based on whether the alert rule relates to positive behavior. Positive behavior may be behavior considered to have a beneficial impact on health. For example, positive behavior may include achieving a number of steps, sleeping a number of hours, achieving a certain resting heart rate, having a certain blood sugar level, or having a certain blood pressure. Decision block 724 may be determined when advisor 702 is configuring the rules rather than after a notification is received by advisor 702.

At block 726, server 706 may send a commendation to client 704. A commendation may be a message praising client 704 for the positive behavior. The message may be an email, text message, push notification, or any suitable message. The commendation may appear to be from advisor 702 rather than user device 606 or an application on user device 606.

At block 728, client 704 may receive the commendation. Client 704 may acknowledge the commendation. client 704 may dismiss the commendation message.

At block 730, the lack of positive behavior may lead to a warning. Advisor 702 may request that the client be warned of the behavior. At block 732, server 706 may send a warning. At block 734, client 704 may receive the warning. Behavior may be continued to be monitored to see if behavior improves. Successive warnings may be sent if behavior does not improve.

At block 736, advisor 702 may request that client 704 schedule an appointment with advisor 702 when the alert rule is not related to positive behavior. Advisor 702 may consider that the alert rule being satisfied requires follow-up. The request for an appointment may follow a warning or may occur immediately after decision block 724.

At block 736, advisor 702 may request the client schedule an appointment. Advisor 702 may wish to see client 704 in order to determine next steps, which may include diagnosis or treatment.

At block 746, advisor 702 may send schedule availability to server 706.

At block 738, server 706 may send the request to schedule an appointment to client 704. The request may be any suitable message, including an email, a text message, a phone call, or a push notification. The request may include information regarding the schedule availability of advisor 702.

At block 740, client 704 may schedule an appointment with advisor 702. client 704 may enter a request for an appointment using user device 606.

At block 742, server 706 may send the appointment to advisor 702.

At block 744, advisor 702 may receive the appointment. Blocks 736 to 744 may be performed in other arrangements as well. For example, client 704 may provide schedule availability to advisor 702, and advisor 702 may schedule the appointment based on the availability of client 704.

Server 706 may save aspects of the communication (e.g., alert rules, sensor data, notifications, commendations, appointments) to datastore 614. These data may be analyzed by analysis engine 616 to generate classifications of the effectiveness of active monitoring programs.

FIG. 8 illustrates the integration of a health monitoring application to a machine learning model. Aspects of the physician application (including blocks 802, 804, 806, 808, and 810) are described in US Patent Publication No. 2023/0230701 A1, entitled “METHODS AND SYSTEMS FOR GENERATING AND MONITORING HOLISTIC TREATMENT PROCESSES”, filed Jan. 20, 2023, the entire contents of which are incorporated herein for all purposes.

Data may include two parts: initial data 802 and symptoms instantiation 804, generated by programs used by patients. Static data may be created by health specialists. Users (e.g., patients) may generate data points during activities. The generated data may be analyzed to determine whether initial data should be modified to improve the process and outcomes for the patient.

Initial data 802 may include a data repository defining programs aimed at management of ailments (e.g., diabetes, heart disease). Programs may include steps divided into two parts: symptoms and actions. Symptoms may be further divided into two parts: holistic and clinical (e.g., “diet recommendation” and “blood pressure”). Symptoms may be defined with a “weight” based on their importance in a specific program. For example, “blood pressure” may have a higher weight in heart disease management than in anxiety management, and fitness may a higher weight in a weight loss program than in a depression program. Holistic steps may be divided into pillars (e.g., Nutrition, Fitness, Mind Health, Supplements, and Care Activities). A successful condition management outcome consists of low level of symptoms and an even performance in all five pillars.

Symptoms instantiation 804 may involve symptoms data points recorded by user (e.g., from a blood pressure monitor) or outputted by a sensor (e.g., a wearable health tracker). Programs defined in initial data may be instantiated by user sessions, and data points related to the programs may be acquired.

At correlation and statistical analysis 806, an algorithm may determine the actions to be performed based on symptom intensity, the type of symptoms, historical data, or the frequency of data points. The algorithm may yield a score for specific actions. Specific actions with a threshold score may be suggested to the user.

At evaluation of progress 808, progress may be indicated by amelioration of symptoms and/or successful and timely execution of recommended actions. A statistical analysis based on previous history and usage frequency may provide progress feedback to the user.

At training 810, the model may be trained for the data points collected. The model may suggest modifications to initial data 802 based on acquired data patterns. For example, initial data 802 may include different standard actions for a set of symptoms. The model may also be trained at an individual level and perform the same functions of suggesting changes to individual program setup.

At advisor portal 812, the trained model may be implemented and/or controllable by a medical practitioner. The medical practitioner may override model training based on their individual considerations or other factors. The medical practitioner may evaluate progress of the user and ramp up or ease off the recommended actions for the user. The progress of the user may be represented graphically so that trends are identifiable. advisor portal 812 may allow medical practitioners to compare outcomes taking into consideration alerts for monitoring various symptoms or actions (e.g., a physician is emailed if blood sugar of a patient exceeds a certain level over a certain period of time). The advisor portal 812 may support combined conditions (e.g., alert if blood pressure is above a certain level and sleep is below a certain level and diet recommendation is not followed or is poorly followed. The absence of data points for a defined period of time (e.g., indicating that monitored user is not using the app) can also be used to trigger alerts. Aspects of advisor portal 812 are described throughout this disclosure, including with FIG. 7.

At portal analysis 814, the effect of active monitoring program with advisor portal 812 of a user may be evaluated compared to the absence of the active monitoring program. Portal analysis 814 may use analysis engine 616. The absence of the active monitoring program may include periodic and manual monitoring of user data by a medical practitioner. The data analyzed may include data from patients who used the active monitoring portal. In some embodiments, the data may include patients who did not use the active monitoring portal. Patient outcomes may be analyzed across different cohorts of users. Cohorts may be grouped by similar demographic information (e.g., age, ethnicity, location, gender), symptom information, treatment information, or other information.

One result of the analysis may be to determine characteristics of patients who had a benefit from an active monitoring program. Analysis for a benefit may be based on comparing patients with and without an active monitoring program, where the patients without an active monitoring program are a control group. Analysis may be based on achieving some measurable improvement in patients with the active monitoring program. For example, analysis may be used to determine types of blood pressure management patients who achieved a certain A1C decrease. The analysis for some measurable improvement may be done with or without a control group. Another result of the analysis may be to determine parameters of the active monitoring program that may lead to a benefit. For example, the analysis may determine the frequency of notifications or the type of notifications for certain disorder management that are effective in improving patient outcomes.

The analysis may be by a statistical model or a machine learning model. Such statistical models may include principal component analysis (PCA), factor analysis (FA), independent component analysis (ICA), multidimensional scaling (MDS), Canonical Correlation Analysis (CCA), Singular Value Decomposition (SVD), and t-Distributed Stochastic Neighbor Embedding (t-SNE). Such machine learning models may include any machine learning model described herein.

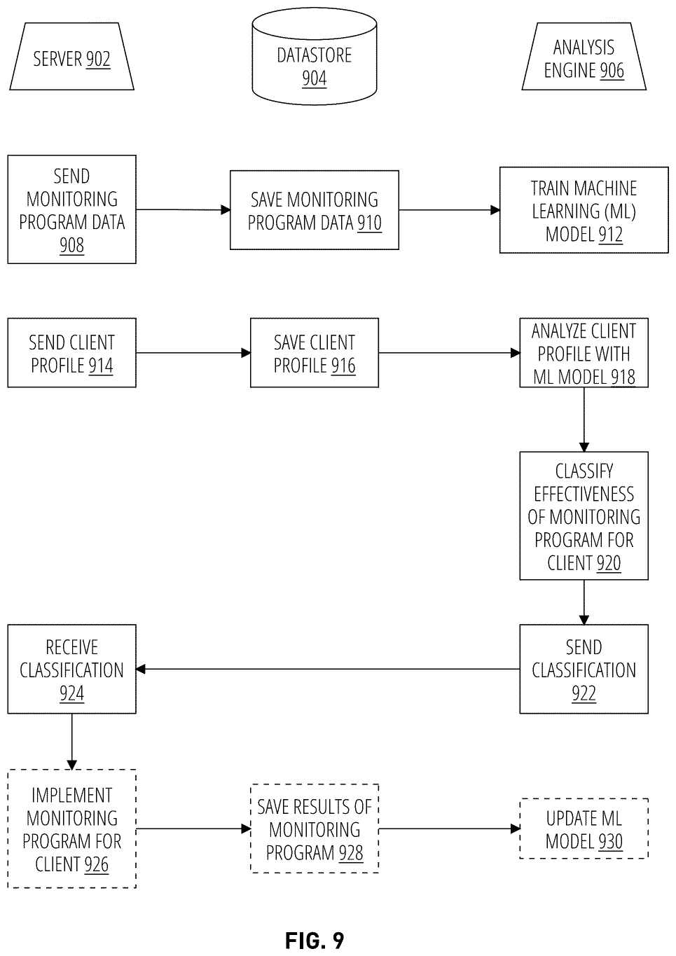

FIG. 9 shows an example of the interaction between server 902, datastore 904, and analysis engine 906. Server 902 may be server 602, server 706, or any server described herein. Datastore 904 may be datastores 614 or any datastore described herein. Analysis engine 906 may be analysis engine 616 or any analysis engine described herein.

At block 908, server 902 may send monitoring program data to datastore 904. Monitoring program data may include data describing clients (e.g., demographics, characteristics, symptoms, treatment plan) and data describing results of the monitoring program (e.g., successful or not successful, quantified measure of success). The monitoring program data may be for a plurality of clients and one or more advisors. At block 910, datastore 904 may save the monitoring program data.

At block 912, analysis engine 906 may use the monitoring program data to train a machine learning (ML) model. The ML model may be machine learning (ML) model(s) 1108 or any machine learning model described herein. The ML model may be trained by using a training data set. The training data set may include data describing the clients and/or the advisors. The training data set may include a set of labels indicating whether a monitoring program was effective for the clients. In some embodiments, the training data set may include a quantifiable measure of the effectiveness (e.g., weight gain/loss, blood pressure, blood sugar level).

Server 902 may receive a client profile. The client profile may include information similar to data for clients in the training data set. For example, the client profile may include demographics, characteristics, symptoms, or treatment plan for the client. In some embodiments, server 902 may receive a profile for the associated advisor of the client. Such a profile for the advisor may include type of advisor, number of clients effectively using the monitoring program, or credentials of the advisor. At block 914, server 902 may send the client profile to datastore 904. An advisor profile may also be sent to datastore 904. At block 916, datastore 904 may save client profile. Datastore 904 may also save the advisor profile.

At block 918, analysis engine 906 may analyze client profile with the ML model. The client profile may be inputted into the ML model. At block 920, analysis engine 906 may classify the effectiveness of the monitoring program for the client. The classification may be an output of the ML model. At block 922, analysis engine 906 may send the classification to server 902.

At block 924, server 902 may receive the classification. At block 926, server 902 may implement the monitoring program for the client. The monitoring program may be implemented only when the classification is that the monitoring program is effective. In some embodiments, the monitoring program may be automatically implemented when the classification indicates the monitoring program is effective. In some embodiments, an advisor may determine that the monitoring program should be implemented. In some instances, the advisor may determine that the monitoring program should not be implemented even when the classification is effective. In other instances, the advisor may determine the monitoring program should be implemented even when the classification is not effective. The advisor may consider a margin of error in the classification determination by analysis engine 906.

At block 928, datastore 904 may save the results of the monitoring program for the client. At block 930, analysis engine 906 may update the ML model using the results. For example, the ML model may undergo additional training with the results from the client and any other clients having the monitoring program implemented since the last training.

In embodiments, datastore 904 may be analyzed for patterns. Datastore 904 may be analyzed with statistical models or ML models to provide heuristics through data aggregation or pattern analysis. Datastore 904 may be analyzed to determine factors that result or generally result in improved outcomes, worse outcomes, or no change in outcomes. Datastore 904 may include data for a plurality of clients and a plurality of advisors.

FIG. 10 is a flowchart of a method 1000 of improving outcomes for a subject. The method may be computer-implemented. The subject may be a patient, someone counseled/coached by an advisor, or any client described herein. Advisors may include healthcare advisors (e.g., physicians, physical therapists, nutritionists, therapists, occupational therapists, nurse practitioners, pharmacists, chiropractors), career and business advisors (e.g., career coaches, mentors, business consultants, recruiters, financial advisors), fitness trainers, educational advisors (e.g., academic advisors, college counselors, guidance counselors, tutors), financial advisors (tax advisors, estate planning advisors, retirement planners), or legal advisors. The method may be performed by a computing system or parts thereof, including server 602, datastore 614, analysis engine 616, server 706, advisor portal 812, portal analysis 814, server 902, datastore 904, machine learning (ML) engine 1104, or computing device 1200.

At block 1002, method 1000 receives, into a model, values of characteristics associated with the subject. The characteristics associated with the subject may include demographic data, symptom data, treatment data, or geographic data. For example, demographic data may include age, race/ethnicity, gender, sexual orientation, education level, income, occupation, marital status, religion, or disability status. Symptom data may include blood pressure, blood sugar level, weight, height, body mass index, body fat percentage, bone density, presence of cough, comfort level, irregular heartbeat, activity level, sleep quantity or quality, or any symptom data described herein. Treatment data may include prescription data (e.g., medication, dosage, frequency), appointment frequency, or exercise regimen. Geographic data may include location of the subject, which may be GPS coordinates, address, neighborhood, city, county, state, or country.

The model may be configured to process data for a plurality of clients and to evaluate an effectiveness of a monitoring program. The plurality of clients may include a monitored subset of clients who have been subject to the monitoring program. The effectiveness of the monitoring program may be evaluated by analyzing outcomes of the monitored subset. For example, the monitoring program may be considered effective if the outcomes are improved from before the start of the monitored program or the outcome is that a certain level is maintained or achieved. The plurality of clients may include an unmonitored subset of clients who have not been subject to the monitoring program. The model may evaluate the effectiveness of a monitoring program by comparing the monitored subset to the unmonitored subset. For example, the model may determine if the outcomes of the monitored subset of clients is statistically different from the outcomes of the unmonitored subset of clients and in a favorable direction.

The monitoring program may be configured to send notifications to one or more advisors when sensor data satisfies an alert rule for a client. The alert rule may be any alert rule described herein. The alert rule may be based on sensor data. Sensor data may include data from any sensor device described herein, including sensor devices 610. Sensor device may include parts of user device 606, including a mobile phone. The mobile phone may generate location, activity, accelerometer data, in addition to collected inputs from the subject. In the non-healthcare context, sensor data may include financial data (e.g., accounts, assets, liabilities, portfolio value), educational data (e.g., test scores, grades), or values of any characteristics described herein. The alert rule may include one or more conditions and one or more actions upon satisfaction of the conditions. The actions may include sending alerts to the client or advisor. The alert rule for the client may be customizable by the one or more advisors. The alert rules may be customized by adjusting the conditions (e.g., changing limits) or by changing the actions.

The monitoring program may be set up for many clients and many advisors. The monitoring program may include a plurality of instructions. The plurality of instructions may include receiving, from the one or more advisors, a plurality of sets of alert rules for the monitored subset of clients. The plurality of instructions may include receiving a plurality of sensor data from a plurality of sensors. The plurality of sensor data may provide information about the monitored subset of clients. The plurality of instructions may include comparing the plurality of sensor data to a plurality of sets of alert rules for the monitored set of clients. The plurality of instructions may include receiving, from the one or more advisors, a plurality of sets of alert rules for the monitored subset of clients. The plurality of sets of alert rules may include different sets of alert rules. Different rules may be for different patients or cohorts of patients.

The model may be a machine learning model. The model may be trained by receiving training data. The training data may include training values of characteristics associated with the plurality of clients and a set of labels indicating the effectiveness of the program for the plurality of clients. The training may include optimizing parameters of the model based on outputs of the model matching or not matching labels of the set of labels when the training values are input into the model. The set of labels indicate the effectiveness of the program for the plurality of clients, and wherein an output of the model specifies whether the monitoring program is effective. The machine learning model may be any machine learning model described herein, including machine learning (ML) model(s) 1108.

In some embodiments, the model may be a statistical model. The statistical models may compare whether a monitoring program is effective for certain values of characteristics for clients. The statistical model may include Principal Component Analysis, Factor Analysis (FA), Independent Component Analysis (ICA), Multidimensional Scaling (MDS), Canonical Correlation Analysis (CCA), Singular Value Decomposition (SVD), or t-Distributed Stochastic Neighbor Embedding (t-SNE). The statistical model may have a certain cutoff for desired amount of improvement in outcome.

At block 1004, method 1000 generates, using the model, a classification of the effectiveness of the monitoring program for the subject. The classification may be a score, with a higher score indicating a more effective or more likely to be effective monitoring program. The classification may include a recommended frequency or type of monitoring for the subject. The type of monitoring may include the specific sensors or alert rules for the monitoring program.

The model may determine the monitoring program is effective for a cohort of clients. The cohort of clients may be distinguished from other patients by having values of characteristics in specific ranges associated with the characteristics. Generating the classification of the effectiveness of the monitoring program may include comparing the values of the characteristics associated with the subject with the values of the characteristics associated with the cohort of clients. The subject may be categorized as being in the cohort of clients. The subject may be categorized by being in the cohort of clients using a vector of characteristics and values of the characteristics. The vector may be compared to a similar vector for the cohort, with cutoffs or ranges for the values of characteristics. The subject may be considered in the cohort if the vector for the subject has a threshold number of characteristics that are within a threshold percentage of the vector for the cohort.

In some embodiments, another machine learning model may determine whether a subject is in a cohort of clients. The machine learning model may be trained on which characteristics and what values make the subject part of that cohort such that the monitoring program has the same or similar effectiveness.

At block 1006, method 1000 inputs a status of the subject into the monitoring program using the classification. The status may indicate whether to implement the monitoring program for the subject.

A selection of a profile for the subject may be received. The profile may include a set of alert rules. The set of alert rules may be tailored for a certain goal, treatment plan, disorder, disease, or other characteristics of the subject. Method 1000 may include receiving, from a target advisor to the subject, a modification to a default set of alert rules for the subject. Modifications may include changing conditions or actions. Modifications may also include deleting alert rules or adding alert rules.

In some embodiments, the status may indicate implementing the monitoring program for the subject. Implementing may include receiving, from a target advisor, a set of subject alert rules. The target advisor may be the advisor associated with the subject. For example, the advisor may be the subject's primary care physician. The subject alert rules may be the default set of alert rules or a modified set or a custom set. Implementing may include receiving sensor data from a sensor. The sensor data may provide information about the subject. Implementing may include comparing sensor data to a set of subject alert rules. Implementing may include sending a notification to the target advisor. Implementing may include receiving, from a target advisor, a set of subject alert rules. The subject alert rules may include determining a time period when sensor data is not being received.

Method 1000 may include sending an initial notification to the subject and a follow-up notification to the subject. Method 1000 may include sending a message commending activity by the subject. Commending the activity may be an initial notification or a follow-up notification. Method 1000 may include receiving, from the subject, a communication granting permission to send notifications for a set of alert rules to a target advisor.

Method 1000 may include receiving a selection of a profile for the subject, wherein the profile includes a set of alert rules. Method 1000 may include receiving, from a target advisor to the subject, a modification to a default set of alert rules for the subject.

A notification may be sent to a target advisor for the subject. The notification may include an option for the target advisor to request the subject to schedule an appointment with the target advisor. A notification may be sent directly to the subject to request an appointment with the target advisor.

In certain embodiments, elements of the client-advisor monitoring system such as alert rules, sensor data categories, notifications, commendations, warnings, and/or appointment requests may be automatically rearranged on a graphical user interface according to defined usage criteria. For example, the system processor may determine the relative frequency with which a patient or advisor interacts with certain alert rules (e.g., blood pressure thresholds, A1C limits, or step count targets) or responds to specific types of notifications. Based on this determination, the system may automatically reposition the most frequently used or most clinically relevant elements so that they are displayed closer to a primary navigation icon, thereby improving accessibility for both clients and advisors and enhancing the effectiveness of active monitoring.

In other embodiments, the rearrangement of elements may be based on a combination of factors, including importance determined by an advisor, prior patient responses, or outcomes associated with particular alerts. For instance, commendations relating to positive behavior (e.g., achieving activity or diet goals) may be elevated in display order if such commendations have historically resulted in improved compliance, while warnings or appointment requests may be prioritized when conditions indicate elevated health risks. The ranking and repositioning of alerts, notifications, or sensor data categories may be updated dynamically over time as the system (e.g., a processor) tracks user interaction patterns and health outcomes, enabling the interface to adapt to evolving patient behavior and advisor guidance. This adaptive arrangement improves over static user interfaces by ensuring that critical elements of monitoring and communication are surfaced to the user in a contextually optimized manner.

Embodiments may include a system including one or more processors. The system may include a non-transitory computer-readable medium storing instructions that when executed by the one or more processors cause the one or more processor to perform any method described herein. Embodiments may also include a non-transitory computer-readable medium storing instructions that when executed by one or more processors, cause the one or more processor to perform any method described herein.

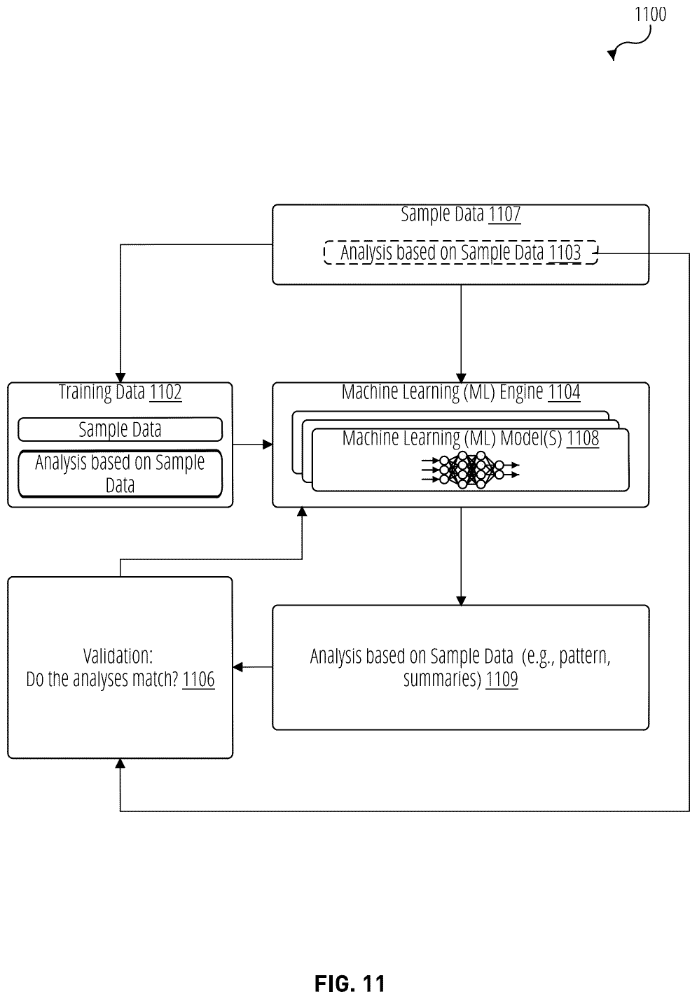

FIG. 11 is a block diagram illustrating using one or more machine learning models 1108 of a machine learning engine 1104 to analyze data to recognize a pattern. The ML engine 1104 generates, trains, and uses the ML model(s) 1108 based using training data 1102. The ML engine 1104 may be analysis engine 616. The ML engine 1104 trains the ML model(s) 1108 to generate an analysis 1109 on input of sample data 1107 into the ML model(s) 1108. The sample data 1107 may include data that is extracted from the data stores (e.g., datastore 614, datastore 904). In some examples, the sample data 1107 may include data that is normalized, merged, and/or processed following extraction (e.g., by any of the systems listed above). In some examples, the sample data 1107 may include some preliminary validation data and/or analysis data, such as summary data (e.g., by any of the systems listed above).

The analysis 1109 output by the ML model(s) 1108 can include at least one pattern identified as part of the analysis 1109 of the sample data 1107. The pattern can include any type of patterns, for instance including patterns associated with high (good) effectiveness and/or patterns associated with low (poor) effectiveness. The analysis 1109 can include a confidence score or score, or a account score or score, as discussed herein. The analysis 1109 can a determination as to an effectiveness of an active monitoring application on a patient.

The training data 1102 that the ML engine 1104 uses to train the ML model(s) 1108 includes sample data (e.g., akin to the sample data 1107) as well as pre-generated assessment(s) corresponding to the sample data (e.g., akin to the analysis 1109 corresponding to the sample data 1107). Over the course of the initial training with training data 1102, the ML model(s) 1108 develop hidden layers between input layers and output layers, and/or weights and/or connections between nodes of the various layers, that each relate to various aspects of the analysis 1109, such as any of the aspects described herein (e.g., related to various types of patterns that can be detected and characteristics of those types of patterns).

In some examples, the ML engine 1104 can continue to train and/or update the ML model(s) 1108 over time, for instance based on validation 1106 using the analysis 1109 and the sample data 1107. In some examples, an analysis 1103 of the sample data 1107 (separate from the analysis 1109 generated by the ML model(s) 1108) may be provided to the ML engine 1104 use in performing the validation 1106. In some examples, the analysis 1103 may be generated by a different entity than the ML model(s) 1108, for instance a different set of ML model(s) (not pictured) or one or more trusted human analysts. If, during validation 1106, the ML engine 1104 determines that the analysis 1109 generated by the ML model(s) 1108 matches the analysis 1103, the ML engine 1104 can treat this as positive feedback, and can perform further training of the ML model(s) 1108 based on the analysis 1109, the sample data 1107, and/or the analysis 1103, for instance to strengthen and/or reinforce weights associated with generating the analysis 1109 in the ML model(s) 1108, and/or to weaken or remove other weights other than those associated with generating the analysis 1109, in the ML model(s) 1108. If, during validation 1106, the ML engine 1104 determines that the analysis 1109 generated by the ML model(s) 1108 differs from the analysis 1103, the ML engine 1104 can treat this as negative feedback, and can perform further training of the ML model(s) 1108 based on the analysis 1109, the sample data 1107, and/or the analysis 1103, for instance to weaken and/or remove weights associated with generating the analysis 1109 in the ML model(s) 1108, and/or to strengthen and/or reinforce other weights other than those associated with generating the analysis 1109 in the ML model(s) 1108.

In some examples, the ML engine 1104 receives feedback during validation 1106 about the analysis 1109. The feedback can include a reaction by a user of a user device via a user interface, a reaction by a user determined based on sensor data from a user device, and/or decisions by a user and/or user device as whether or not to use the analysis 1109 for a further application. Positive feedback can be used to strengthen and/or reinforce weights associated with generating the analysis 1109 in the ML model(s) 1108, and/or to weaken or remove other weights other than those associated with generating the analysis 1109 in the ML model(s) 1108. Negative feedback can be used to weaken and/or remove weights associated with generating the analysis 1109 in the ML model(s) 1108, and/or to strengthen and/or reinforce other weights other than those associated with generating the analysis 1109 in the ML model(s) 1108.

The ML engine 1104, the ML model(s) 1108, and/or the ML model(s) 1425 can include one or more neural network (NNs), one or more convolutional neural networks (CNNs), one or more trained time delay neural networks (TDNNs), one or more deep networks, one or more autoencoders, one or more deep belief nets (DBNs), one or more recurrent neural networks (RNNs), one or more generative adversarial networks (GANs), one or more conditional generative adversarial networks (cGANs), one or more other types of neural networks, one or more trained support vector machines (SVMs), one or more trained random forests (RFs), one or more computer vision systems, one or more deep learning systems, one or more classifiers, one or more transformers, or combinations thereof. Within FIG. 11, a graphic representing the trained machine learning (ML) model(s) 1108 is illustrated as a set of circles connected to another. Each of the circles can represent a node, a neuron, a perceptron, a layer, a portion thereof, or a combination thereof. The circles are arranged in columns. The leftmost column of white circles represent an input layer. The rightmost column of white circles represent an output layer. Two columns of shaded circled between the leftmost column of white circles and the rightmost column of white circles each represent hidden layers. The ML engine 1104 and/or the ML model(s) 1108 can be part of any AI and/or ML modules, processes, or analysis operations discussed herein.

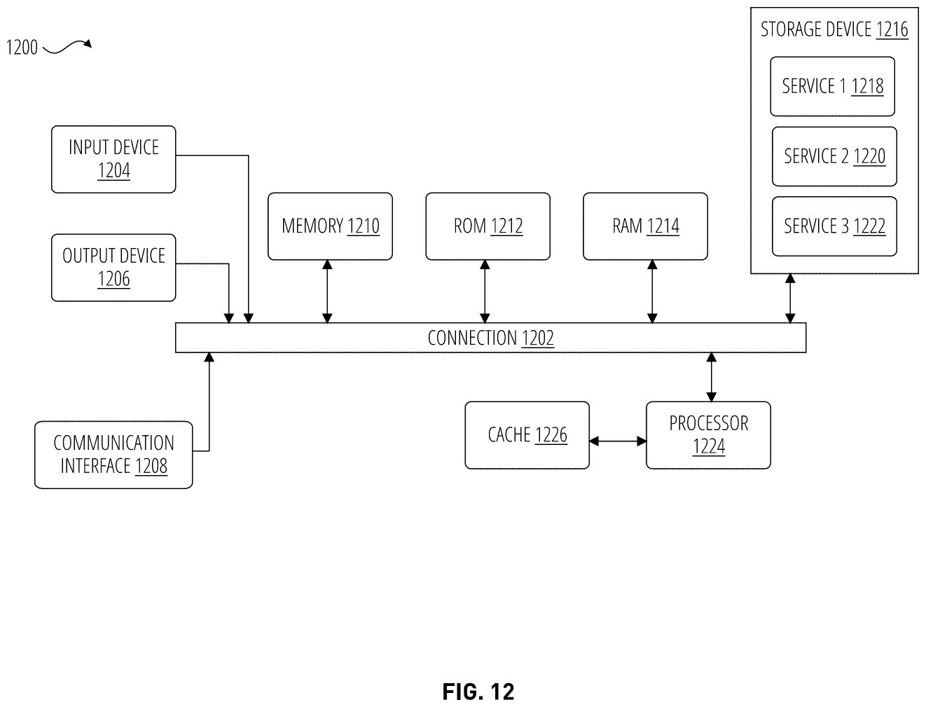

FIG. 12 illustrates an example computing device according to aspects of the present disclosure. For example, computing device 1200 can implement any of the systems or methods described herein. In some instances, computing device 1200 may be a component of or included within a media device. The components of computing device 1200 are shown in electrical communication with each other using connection 1202, such as a bus. The example computing device 1200 includes a processor 1224 (e.g., CPU, processor, or the like) and connection 1202 (e.g., such as a bus, or the like) that is configured to couple components of computing device 1200 such as, but not limited to, memory 1210, read only memory (ROM) 1212, random access memory (RAM) 1214, and/or storage device 1216, to processor 1224.

Computing device 1200 can include a cache 1226 of high-speed memory connected directly with, in close proximity to, or integrated within processor 1224. Computing device 1200 can copy data from memory 1210 and/or storage device 1216 to cache 1226 for quicker access by processor 1224. In this way, cache 1226 may provide a performance boost that avoids delays while processor 1224 waits for data. Alternatively, processor 1224 may access data directly from memory 1210, ROM 1212, ram 1214, and/or storage device 1216. Memory 1210 can include multiple types of homogenous or heterogeneous memory (e.g., such as, but not limited to, magnetic, optical, solid-state, etc.).

Storage device 1216 may include one or more non-transitory computer-readable media such as volatile and/or non-volatile memories. A non-transitory computer-readable medium can store instructions and/or data accessible by computing device 1200. Non-transitory computer-readable media can include, but is not limited to magnetic cassettes, hard-disk drives (HDD), flash memory, solid state memory devices, digital versatile disks, cartridges, compact discs, random access memories (RAMs) 1214, read only memory (ROM) 1212, combinations thereof, or the like.

Storage device 1216, may store one or more services, such as service 1 1218, service 2 1220, and service 3 1222, that are executable by processor 1224 and/or other electronic hardware. The one or more services include instructions executable by processor 1224 to: perform operations such as any of the techniques, steps, processes, blocks, and/or operations described herein; control the operations of a device in communication with computing device 1200; control the operations of processor 1224 and/or any special-purpose processors; combinations therefor; or the like. Processor 1224 may be a system on a chip (SOC) that includes one or more cores or processors, a bus, memories, clock, memory controller, cache, other processor components, and/or the like. A multi-core processor may be symmetric or asymmetric.

Computing device 1200 may include one or more input devices 1204 that may represent any number of input mechanisms, such as a microphone, a touch-sensitive screen for graphical input, keyboard, mouse, motion input, speech, media devices, sensors, combinations thereof, or the like. Computing device 1200 may include one or more output devices 1206 that output data to a user. Such output devices 1206 may include, but are not limited to, a media device, projector, television, speakers, combinations thereof, or the like. In some instances, multimodal computing devices can enable a user to provide multiple types of input to communicate with computing device 1200. Communications communication interface 1208 may be configured to manage user input and computing device output. Communications communication interface 1208 may also be configured to managing communications with remote devices (e.g., establishing connection, receiving/transmitting communications, etc.) over one or more communication protocols and/or over one or more communication media (e.g., wired, wireless, etc.).

Computing device 1200 is not limited to the components as shown in FIG. 12. Computing device 1200 may include other components not shown and/or components shown may be omitted.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order that is logically possible. Additionally, portions of these steps may be used with portions of other steps from other methods. Also, all or portions of a step may be optional. Additionally, any of the steps of any of the methods can be performed with modules, units, circuits, or other means of a system for performing these steps.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The above description of example embodiments of the present disclosure has been presented for the purposes of illustration and description and are set forth so as to provide those of ordinary skill in the art with a complete disclosure and description of how to make and use embodiments of the present disclosure. It is not intended to be exhaustive or to limit the disclosure to the precise form described nor are they intended to represent that the experiments are all or the only experiments performed. Although the disclosure has been described in some detail by way of illustration and example for purposes of clarity of understanding, it is readily apparent to those of ordinary skill in the art in light of the teachings of this disclosure that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the disclosure being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

All patents, patent applications, publications, and descriptions mentioned herein are hereby incorporated by reference in their entirety for all purposes as if each individual publication or patent were specifically and individually indicated to be incorporated by reference and are incorporated herein by reference to disclose and describe the methods and/or materials in connection with which the publications are cited. None is admitted to be prior art.

What is claimed is:

1. A computer-implemented method, comprising:
receiving, into a model, values of characteristics associated with a subject, wherein the model is configured to process data for a plurality of clients and to evaluate an effectiveness of a monitoring program, wherein the plurality of clients includes a monitored subset of clients who have been subject to the monitoring program, wherein the monitoring program is configured to send notifications to one or more advisors when sensor data satisfies an alert rule for a client, and wherein the alert rule for the client is customizable by the one or more advisors;
generating, using the model, a classification of the effectiveness of the monitoring program for the subject; and
inputting a status of the subject into the monitoring program using the classification, wherein the status indicates whether to implement the monitoring program for the subject.

2. The computer-implemented method of claim 1, wherein:
the plurality of clients includes an unmonitored subset of clients who have not been subject to the monitoring program.

3. The computer-implemented method of claim 1, wherein:
the status indicates implementing the monitoring program for the subject, and
implementing includes comparing sensor data to a set of subject alert rules.

4. The computer-implemented method of claim 1, wherein:
the status indicates implementing the monitoring program for the subject, and
implementing includes sending a notification to a target advisor.

5. The computer-implemented method of claim 1, wherein:
the status indicates implementing the monitoring program for the subject,
implementing includes receiving, from a target advisor, a set of subject alert rules, and
a subject alert rule includes determining a time period when the sensor data is not being received.

6. The computer-implemented method of claim 1, wherein:
the model is a machine learning model,
the model is trained by receiving training data, and
the training data includes training values of characteristics associated with the plurality of clients and a set of labels indicating the effectiveness of the program for the plurality of clients.

7. The computer-implemented method of claim 1, wherein the model is a machine learning model, and wherein the model is trained by:
optimizing parameters of the model based on outputs of the model matching or not matching labels of a set of labels when training values are input into the model, wherein the set of labels indicate the effectiveness of the program for the plurality of clients, and wherein an output of the model specifies whether the monitoring program is effective.

8. The computer-implemented method of claim 1, wherein:
the model determines the monitoring program is effective for a cohort of clients;
the cohort of clients is distinguished from other patients by having values of characteristics in specific ranges associated with the characteristics; and
generating the classification of the effectiveness of the monitoring program includes comparing the values of the characteristics associated with the subject with the values of the characteristics associated with the cohort of clients.

9. The computer-implemented method of claim 1, wherein:
the model determines the monitoring program is effective for a cohort of patients;
the cohort of patients is distinguished from other patients by having values of characteristics in specific ranges associated with the characteristics; and
generating the classification of the effectiveness of the monitoring program includes determining the subject is categorized as being in the cohort of patients.

10. The computer-implemented method of claim 1, wherein:
the monitoring program includes a plurality of instructions, and
the plurality of instructions includes receiving, from the one or more advisors, a plurality of sets of alert rules for the monitored subset of clients.

11. The computer-implemented method of claim 1, wherein:
the monitoring program includes a plurality of instructions,
the plurality of instructions includes receiving a plurality of sensor data from a plurality of sensors, and
the plurality of sensor data provides information about the monitored subset of clients.

12. The computer-implemented method of claim 1, wherein:
the monitoring program includes a plurality of instructions, and
the plurality of instructions includes comparing the plurality of sensor data to a plurality of sets of alert rules for the monitored subset of clients.

13. The computer-implemented method of claim 1, wherein:
the monitoring program includes a plurality of instructions,
the plurality of instructions includes receiving, from the one or more advisors, a plurality of sets of alert rules for the monitored subset of clients, and
the plurality of sets of alert rules includes different sets of alert rules.

14. The computer-implemented method of claim 1, wherein:
the monitoring program includes a plurality of instructions, and
the plurality of instructions includes receiving, from the one or more advisors, an instruction for whether to send a follow-up notifications to the client after an initial notification to the client.

15. The computer-implemented method of claim 1, further comprising:
implementing the monitoring program for the subject, and
sending an initial notification and a follow-up notification to the subject.

16. The computer-implemented method of claim 1, further comprising:
implementing the monitoring program for the subject, and
sending a message commending activity by the subject.

17. The computer-implemented method of claim 1, further comprising:
receiving a selection of a profile for the subject, wherein the the profile includes a set of alert rules.

18. The computer-implemented method of claim 1, further comprising:
receiving, from a target advisor to the subject, a modification to a default set of alert rules for the subject.

19. A system comprising:
one or more processors; and
a non-transitory computer-readable medium storing instructions that when executed by the one or more processors cause the one or more processor to perform a method comprising:
receiving, into a model, values of characteristics associated with a subject, wherein the model is configured to process data for a plurality of clients and to evaluate an effectiveness of a monitoring program, wherein the plurality of clients includes a monitored subset of clients who have been subject to the monitoring program, wherein the monitoring program is configured to send notifications to one or more advisors when sensor data satisfies an alert rule for a client, and wherein the alert rule for the client is customizable by the one or more advisors;
generating, using the model, a classification of the effectiveness of the monitoring program for the subject; and
inputting a status of the subject into the monitoring program using the classification, wherein the status indicates whether to implement the monitoring program for the subject.

20. A non-transitory computer-readable medium storing instructions that when executed by one or more processors, cause the one or more processor to perform a method comprising:
receiving, into a model, values of characteristics associated with a subject, wherein the model is configured to process data for a plurality of clients and to evaluate an effectiveness of a monitoring program, wherein the plurality of clients includes a monitored subset of clients who have been subject to the monitoring program, wherein the monitoring program is configured to send notifications to one or more advisors when sensor data satisfies an alert rule for a client, and wherein the alert rule for the client is customizable by the one or more advisors;
generating, using the model, a classification of the effectiveness of the monitoring program for the subject; and
inputting a status of the subject into the monitoring program using the classification, wherein the status indicates whether to implement the monitoring program for the subject.
